(19) United States
(12) Patent Application Publication
(10) Pub. No.: US 2025/0052744 A1
(43) Pub. Date: Feb. 13, 2025

(54) Dyes and Uses Thereof

(71) Applicant: The University of Liverpool, Liverpool (GB)

(72) Inventor: Srijan Jindal, Oxford (GB)

(21) Appl. No.: 18/722,913

(22) PCT Filed: Dec. 23, 2022
(86) PCT No.: PCT/GB2022/053397

§ 371 Date: Jun. 21, 2024

(30) Foreign Application Priority Data
Dec. 23, 2021 (GB) 2118957.6

(51) Int. Cl. G01N 33/53; C09K 11/06; G01N 33/569

(52) U.S. Cl. CPC G01N 33/5308; C09K 11/06; G01N 33/56911

(57) ABSTRACT

The present invention relates to a combination of fluorescent dyes for identifying, or differentiating between, different bacterial strains, wherein the combination comprises two or more of the following fluorescent dyes: SYBR Green I, SYTO 13 or 9, and/or DiSC3(5) and/or FM4-64. The device is particularly suited for identifying the type of bacterial infection causing a Urinary Tract Infection (UTI) and can be used to help identify which antimicrobial agents would be suitable for the treatment the infection.

### TECHNICAL FIELD OF THE INVENTION

This invention relates to dyes for use in identifying and/or differentiating bacterial strains and species. The invention is useful in rapid assays for identifying and/or differentiating bacterial strains and species in biological samples. The dyes are particularly suited for assessing antimicrobial susceptibility in biological samples from patients suspected of suffering from a microbial infection, and in particular for detecting antimicrobial susceptibility of bacteria in urine samples from patients suffering a Urinary Tract Infection (“UTI”).

### BACKGROUND TO THE INVENTION

Progress in the development of medical devices used for testing patient samples has accelerated in recent years due to advances in computing and the miniaturisation of electrical components. However, medical devices deployed in point of care settings still present an issue as the patient sample typically must be processed a certain way in order to prepare the sample before being presented to, and correctly analysed by, the device. The patient sample may be processed by adding the sample to reagents in a mixing vessel to form a mixture before presenting the mixture to the device for analysis. Frequently, the patient sample is not processed correctly by the healthcare professional. This either renders the sample unusable for analysis (requiring further samples to be taken from the patient) or can result in the wrong or incomplete diagnosis being given by the device. Furthermore, the act of mixing patient samples with reagents can pose a biohazard threat to the healthcare professional if adequate personal protective equipment is not (or incorrectly) deployed.

There are a number of medical conditions where a quick and reliable point of care medical device could make a dramatic difference to the correct diagnosis and result in the prescription of the correct medicine the first time. Microbial infections is one such area, where a physician often prescribes a course of a particular antimicrobial drug, despite not knowing whether the infection is of viral or bacterial origin and indeed whether it is a bacterial infection to which the antimicrobial drug is effective. The patient may return to physician after the course of antimicrobials if they have not worked, where the physician will then proceed to prescribe a different course of antimicrobials which may be effective. This approach not only wastes the time of both the patient and physician, but puts the patient at risk of the infection worsening and also contributes to the rise of resistance to antimicrobials.

What would be desirable would be a very rapid means of knowing, even before a patient left a doctor's surgery, that a particular antimicrobial was indeed capable of killing the organism causing the infection. While genotypic (whole-genome-sequencing) methods hold out some promise for this, what is really desired is a phenotypic assay that assesses the activity of anti-infectives in the sample itself.

Urinary tract infections (“UTIs”) are a worldwide patient problem. Other than in hospital-acquired infections, they are particularly common in females, with 1 in 2 women experiencing a UTI at some point in their life. Escherichia coli is the most common causative pathogen of a UTI. However, other Enterobacteriaceae such as Proteus mirabilis, Klebsiella spp. and Pseudomonas aeruginosa, and even Gram-positive cocci such as staphylococci and enterococci, may also be found (Kline and Lewis 2016; Tandogdu and Wagenlehner 2016).

E. coli cells in all conditions are highly heterogeneous (Kell et al. 2015), even if only because they are in different phases of the cell cycle (Wallden et al. 2016), and in both ‘exponential’ and stationary phase contain a variety of chromosome numbers (Åkerlund et al. 1995; Skarstad et al. 1986; Skarstad et al. 1985; Steen and Boye 1980; Stokke et al. 2012). To discriminate them physiologically, and especially to relate them to culturability (a property of an individual), it is necessary to study them individually (Kell et al. 1991; Taheri-Araghi et al. 2015), typically using flow cytometry. Flow cytometry has also been used to count microbes (and indeed white blood cells) for the purposes of assessing UTIs. Single cell morphological imaging has also been used, where in favourable cases antibiotic susceptibility can be detected in 15-30 minutes or less (Baltekin et al. 2017; Choi et al. 2014).

WO2020/109764 discloses method for rapidly determining the susceptibility of a microorganism to an antimicrobial agent using flow cytometry. Whilst the method was successful in quickly determining the susceptibility of a microorganism to an antimicrobial agent, flow cytometry is currently expensive and requires complex instrumentation and a significant amount of reagents to evaluate cell growth.

An object of the present invention is to provide dyes which can be used in rapid assays for use in identifying and/or differentiating bacterial strains and species in biological samples. It is desirable that a single mixture of dyes can be used to test for a number of different bacterial strains and species so as to facilitate a fast and accurate assay which would enable a healthcare worker or physician to quickly assess which bacterial strain or species is causing an infection in a patient and allow the prescription of an antimicrobial therapy that would successfully treat the bacterial infection. It would also be desirable if the dyes were able to identify and/or differentiate bacterial strains and species in urine from patients suffering from UTIs.

### SUMMARY OF THE INVENTION

In accordance with a first aspect of the present invention, there is provided a combination of fluorescent dyes for identifying, or differentiating between, different bacterial strains, wherein the combination comprises two or more DNA staining fluorescent dyes and/or a membrane staining dye and/or a cellular accumulation dye and wherein the two or more DNA staining dyes and/or a cellular accumulation dye and/or membrane dye are selected from the following fluorescent dyes: SYBR Green I, SYTO 13 or 9, DiSC3(5), and/or FM4-64.

The combination of fluorescent dyes of the present invention is relative simple to produce and enables the bacteria in various biological samples to be quickly identified. It also enables multiple assays to be run simultaneously on the same biological sample or on different samples. Additionally, the combination can be used in conjunction with assays including and omitting antimicrobial agents so as to allow the susceptibility of the bacterial strains to be quickly established. In a clinical setting, this would enable the physician to quickly identify which antimicrobial agent would be most successful in treating a particular infection.

It is preferred that the combination comprises SYBR Green I, SYTO 13 or 9, and DiSC3(5). The SYTO 13 or 9 may be present in a substantially similar amount to DiSC3(5). In one embodiment, the SYBR Green I is present in an amount of about 10×, SYTO 13 or 9 is present in an amount of about 3 μM and DiSC3(5) is present in an amount of about 3 μM. SYBR Green I is delivered as a 10,000× concentration to be used at 1× for staining DNA. Usually SYBR Green is used at 1 μM for staining DNA so 10× would roughly be about 10 μM.

In certain embodiments, the ratio of SYBR Green I to SYTO 13 or 9 to DiSC3(5) may be about 10 to about 3 to about 3. The present inventors tested numerous concentrations for each of the dyes and this ratio was advantageously found to not saturate the fluorescence levels, whilst still being visible using a fluorescent microscope.

It is preferred that the combination further comprises FM 4-64 in an amount of about 15 μM.

SYBR Green I is an asymmetrical cyanine dye used as a nucleic acid stain (N′,N′-dimethyl-N-[4-[(E)-(3-methyl-1,3-benzothiazol-2-ylidene)methyl]-1-phenylquinolin-1-ium-2-yl]-N-propylpropane-1,3-diamine). SYTO 13 or 9 are both green fluorescent nucleic acid stains. DiSC3(5) is a cationic carbocyanine with a short (C3) alkyl tail (3,3′-Dipropylthiadicarbocyanine Iodide). FM™ 4-64 is a lipophilic dye which is used for visualising yeast vacuolar membranes (N-(3-Triethylammoniumpropyl)-4-(6-(4-(Diethylamino) Phenyl) Hexatrienyl) Pyridinium Dibromide).

The combination may be incorporated into a bacterial growth media.

The combination may be added to a biological sample. The biological sample will preferably be derived from an individual believed to be suffering from a microorganism infection. The biological sample may be derived directly from potentially any body fluid, such as urine, blood, mucus or saliva. The biological samples may be whole or pre-treated with reagents or buffers or filtered. In certain embodiments, the biological sample is urine.

The combination may be coated or provided in the chamber of a device for determining the susceptibility of a microorganism in an aqueous biological sample to an antimicrobial agent.

In accordance with a second aspect of the present invention, there is provided a method for identifying, or differentiating between, different bacterial strains in a biological sample, the method comprising:
a) contacting a dye combination of two of more DNA staining fluorescent dyes and/or a cellular accumulation dye and/or a membrane dye with an aqueous biological sample wherein the two or more DNA staining dyes and/or a cellular accumulation dye and/or membrane staining dye are selected from the following fluorescent dyes: SYBR Green I, SYTO 13 or 9, DiSC3(5), and/or FM4-64;
b) incubating the dye combination and the biological sample under conditions effective to enable staining of bacterial cells;
c) taking at least one image of the stained bacterial cells; and
d) analysing the at least one image for fluorescent signal intensities for each of the two of more fluorescent dyes and comparing the fluorescent signal intensities with that of known fluorescent signal intensities for previously screened bacterial strains and identifying the bacterial strain in the biological sample when the fluorescent signal intensities are substantially similar to the fluorescent signal intensities of one of the previously screened bacterial strains.

The term, “aqueous biological sample” is intended to mean a sample derived from (or is) any bodily fluid (such as urine, blood, mucus or saliva for example) which includes a component of water which is available for the hydration of the hydrogel.

The combination preferably comprises SYBR Green I, SYTO 13 or 9, and DiSC3(5).

In the method, it is preferred that the combination comprises SYBR Green I, SYTO 13 or 9, and DiSC3(5). The SYTO 13 or 9 may be present in a substantially similar amount to DiSC3(5). In one embodiment, the SYBR Green I is present in an amount of about 10× (or 10 μM), SYTO 13 or 9 is present in an amount of about 3 μM and DiSC3(5) is present in an amount of about 3 μM. The ratio of SYBR Green I to SYTO 13 or 9 to DiSC3(5) may be about 10 to about 3 to about 3.

If the combination comprises FM 4-64, it is preferred that it is present in an amount of about 15 μM.

The dye combination may be incorporated into a bacterial growth media.

In one embodiment, the dye combination is in a desiccated form prior to being contacted with biological sample.

Step b) may further comprise incubating the biological sample in growth media under conditions effective to enable or encourage growth or proliferation of the bacterial cells.

Step b) may further comprises heating the biological sample in the range of about 35° C. and about 40° C. and preferably at a temperature of about 37° C. The incubation step b) may be up to about 1 hour, up to about 55 minutes, up to about 50 minutes, up to about 45 minutes, up to about 40 minutes, up to about 35 minutes, up to about 30 minutes, up to about 25 minutes, up to about 20 minutes or up to about 10 minutes.

The biological sample may be derived from an individual believed to be suffering from a bacterial infection and the biological sample may be urine. The bacterial infection may be a Urinary Tract Infection (UTI).

The stained bacterial cells will preferably be imaged using a microscope. The microscope may additionally comprise or incorporate filters. Such filters could be either optical filters or software filters applied to the image data or a combination of both.

Step c) may comprise taking images of the stained bacterial cells continuously or periodically during incubation.

The method may additionally comprise identifying Enterococcus spp. in a biological sample using the relative fluorescent intensity and percentage of bacterial cells stained for the following dyes: SYBR Green I, DiSC3(5), FM4-64 and SYTO 13. In a related embodiment the relative fluorescent intensity of SYBR Green I is medium to high, the relative fluorescent intensity of DiSC3(5) is low, the relative fluorescent intensity of FM4-64 is medium, the relative fluorescent intensity of SYTO 13 is medium. In a further related embodiment the percentage of bacterial cells stained in SYBR Green I is almost all (high), the percentage of bacterial cells stained in DiSC3(5) is about half, the percentage of bacterial cells stained in FM4-64 is almost all, the percentage of bacterial cells stained in SYTO 13 is almost all. The method may additionally comprise identifying Staphylococcus aureus in a biological sample using the relative fluorescent intensity and percentage of bacterial cells stained for the following dyes: SYBR Green I and DiSC3(5). In a related embodiment the relative fluorescent intensity of SYBR Green I is high, the relative fluorescent intensity of DiSC3(5) is high. In a further related embodiment the percentage of bacterial cells stained in SYBR Green I is almost all, the percentage of bacterial cells stained in DiSC3(5) is almost all.

The method may additionally comprise identifying Staphylococcus epidermis in a biological sample using the relative fluorescent intensity and percentage of bacterial cells stained for the following dyes: DiSC3(5) and SYTO 13. In a related embodiment the relative fluorescent intensity of DiSC3(5) is very high, the relative fluorescent intensity of SYTO 13 is high. In a further related embodiment the percentage of bacterial cells stained in DiSC3(5) is almost all, the percentage of bacterial cells stained in SYTO 13 is almost all.

The method may additionally comprise identifying Staphylococcus saprophyticus in a biological sample using the relative fluorescent intensity and percentage of bacterial cells stained for the following dyes: DiSC3(5) and SYTO 13. In a related embodiment the relative fluorescent intensity of DiSC3(5) is high, the relative fluorescent intensity of SYTO 13 is medium. In a further related embodiment the percentage of bacterial cells stained in DiSC3(5) is almost all, the percentage of bacterial cells stained in SYTO 13 is almost all.

The method may additionally comprise identifying Streptococcus agalactiae in a biological sample using the relative fluorescent intensity and percentage of bacterial cells stained for the following dyes: SYBR Green I and DiSC3(5). In a related embodiment the relative fluorescent intensity of SYBR Green I is very high, the relative fluorescent intensity of DiSC3(5) is low. In a further related embodiment the percentage of bacterial cells stained in SYBR Green I is almost all, the percentage of bacterial cells stained in DiSC3(5) is almost all.

The method may additionally comprise identifying Escherichia coli in a biological sample using the relative fluorescent intensity and percentage of bacterial cells stained for the following dyes: SYBR Green I, DiSC3(5), FM4-64 and SYTO 13. In a related embodiment the relative fluorescent intensity of SYBR Green I is medium, the relative fluorescent intensity of DiSC3(5) is medium, the relative fluorescent intensity of FM4-64 is medium, the relative fluorescent intensity of SYTO 13 is medium. In a further related embodiment the percentage of bacterial cells stained in SYBR Green I is almost all, the percentage of bacterial cells stained in DiSC3(5) is almost all, the percentage of bacterial cells stained in FM4-64 is almost all, the percentage of bacterial cells stained in SYTO 13 is almost all.

The method may additionally comprise identifying Klebsiella in a biological sample using the relative fluorescent intensity and percentage of bacterial cells stained for the following dyes: SYBR Green I, DiSC3(5), FM4-64 and SYTO 13. In a related embodiment the relative fluorescent intensity of SYBR Green I is low, the relative fluorescent intensity of DiSC3(5) is low, the relative fluorescent intensity of FM4-64 is low, the relative fluorescent intensity of SYTO 13 is low. In a further related embodiment the percentage of bacterial cells stained in SYBR Green I is few (low), the percentage of bacterial cells stained in DiSC3(5) is few, the percentage of bacterial cells stained in FM4-64 is few, the percentage of bacterial cells stained in SYTO 13 is few.

The method may additionally comprise identifying Proteus in a biological sample using the relative fluorescent intensity and percentage of bacterial cells stained for the following dyes: FM4-64. In a related embodiment the relative fluorescent intensity of FM4-64 is negligible. In a further related embodiment the percentage of bacterial cells stained in FM4-64 is few.

The method may additionally comprise identifying Pseudomonas aeruginosa in a biological sample using the relative fluorescent intensity and percentage of bacterial cells stained for the following dyes: DiSC3(5). In a related embodiment the relative fluorescent intensity of DiSC3(5) is negligible. In a further related embodiment the percentage of bacterial cells stained in DiSC3(5) is few.

In accordance with a third aspect of the present invention, there is provide a kit for identifying, or differentiating between, different bacterial strains, in an aqueous biological sample, the kit comprising:
a) a combination of fluorescent dyes wherein the combination comprises two of more DNA staining fluorescent dyes and/or a cellular accumulation dye and/or a membrane staining dye wherein the two or more DNA staining dyes and/or a cellular accumulation dye and/or membrane staining dye are selected from the following fluorescent dyes: SYBR Green I, SYTO 13 or 9, DiSC3(5), and/or FM4-64;
b) at least one chamber for enabling the mixing of the dye combination with the biological sample so as to form a mixture;
c) an imaging device for taking at least one image of the stained bacterial cells in the mixture and analysing the at least one image for fluorescent signal intensities for each of the two of more fluorescent dyes and comparing the fluorescent signal intensities with that of known fluorescent signal intensities for previously screened bacterial strains and identifying the bacterial strain in the biological sample when the fluorescent signal intensities are substantially similar to the fluorescent signal intensities of one of the previously screened bacterial strainsbacterial strain.

It is preferred that the combination comprises SYBR Green I, SYTO 13 or 9, and DiSC3(5). The SYTO 13 or 9 may be present in a substantially similar amount to DiSC3(5). In one embodiment, the SYBR Green I is present in an amount of about 10× (or 10 μM), SYTO 13 or 9 is present in an amount of about 3 μM and DiSC3(5) is present in an amount of about 3 μM. The ratio of SYBR Green I to SYTO 13 or 9 to DiSC3(5) may be about 10 to about 3 to about 3.

If the kit comprises FM 4-64, it is preferred that it is present in an amount of about 15 μM.

The dye combination may be in a desiccated or dried form.

The chamber may be coated in the dye combination. The kit may additionally comprise at least one transparent covers to cover the at least one chamber.

The chamber may be filled with the dye combination, and optionally, other reagents and/or buffers.

The kit may further comprise growth media.

The kit may further comprise a heating arrangement for heating the chamber.

In certain embodiments, the kit may further comprise two or more chambers.

The kit may further comprises one or more antimicrobial agents.

The kit may be for use in the method herein above described with reference to the second aspect.

Features, integers, characteristics, compounds, described in conjunction with a particular aspect, embodiment or example of the invention are to be understood to be applicable to any other aspect, embodiment or example described herein unless incompatible therewith. All of the features disclosed in this specification (including any accompanying claims, abstract and figures), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

### DETAILED DESCRIPTION OF THE INVENTION

Embodiments of the invention are described below, by way of example only, with reference to the accompanying figure in which:

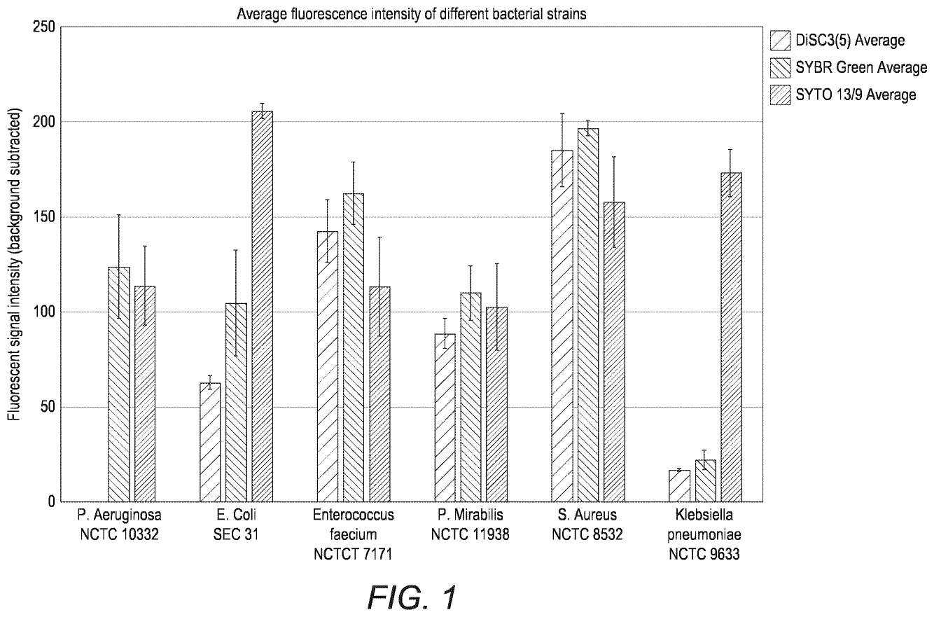

FIG. 1 is a graph showing the fluorescent signal intensity value of P. Aeruginosa NCTC 10332, E. Coli SEC 31, Enterococcus faecium NCTCT 7171, P. Mirabilis NCTC 11938, S. Aureus NCTC 8532 and Klebsiella pneumoniae NCTC 9633, corresponding to DiSC3(5), SYBR Green I and SYTO 13/9 dyes.

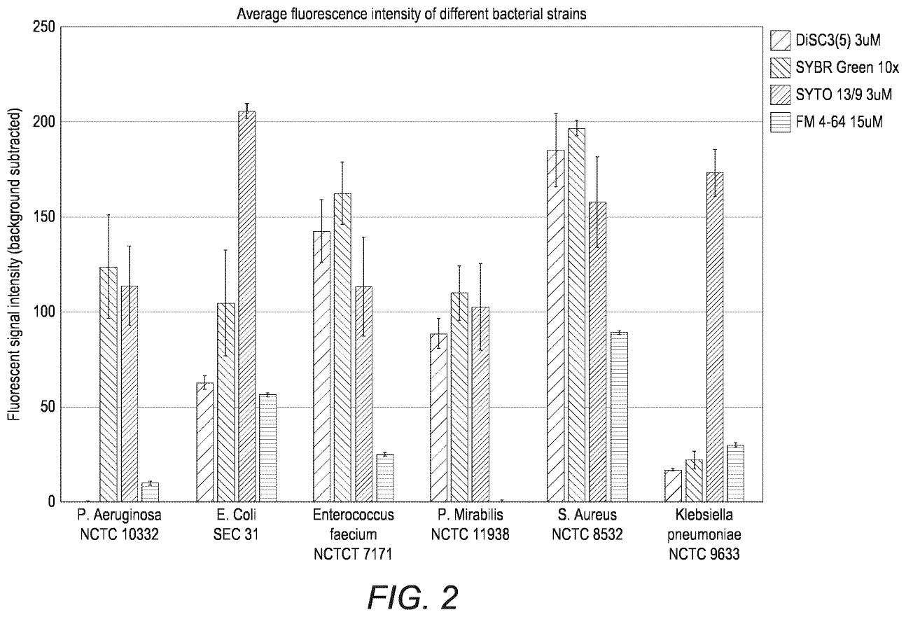

FIG. 2 is a graph showing the fluorescent signal intensity value of P. Aeruginosa NCTC 10332, E. Coli SEC 31, Enterococcus faecium NCTCT 7171, P. Mirabilis NCTC 11938, S. Aureus NCTC 8532 and Klebsiella pneumoniae NCTC 9633, corresponding to DiSC3(5), SYBR Green I, SYTO 13/9 and FM4-64 dyes.

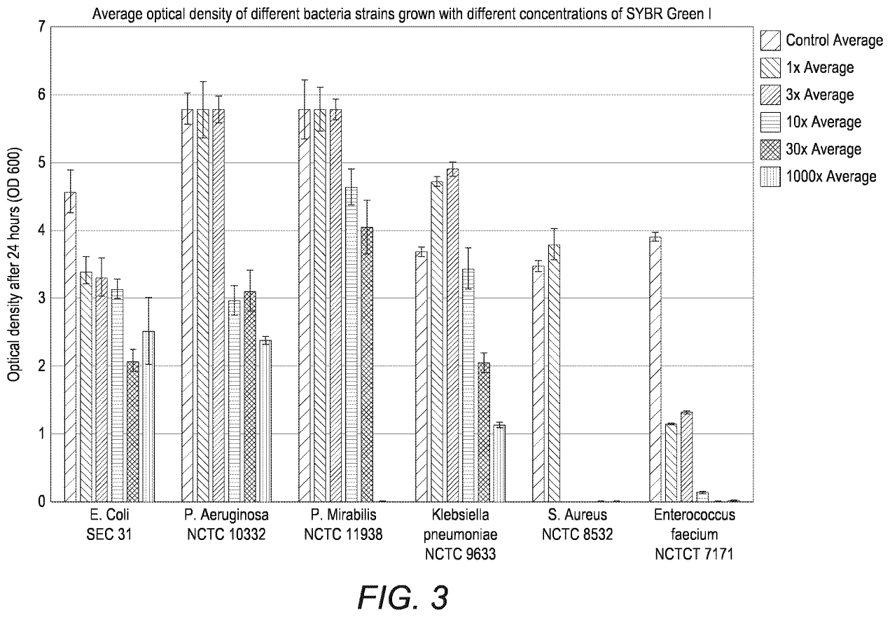

FIG. 3 is a graph showing is a graph showing the average optical density after 24 hours (OD 600) of P. Aeruginosa NCTC 10332, E. Coli SEC 31, Enterococcus faecium NCTCT 7171, P. Mirabilis NCTC 11938, S. Aureus NCTC 8532 and Klebsiella pneumoniae NCTC 9633, when incubated with DiSC3(5), SYBR Green I and SYTO 13/9 where different concentrations of SYBR Green I were assessed.

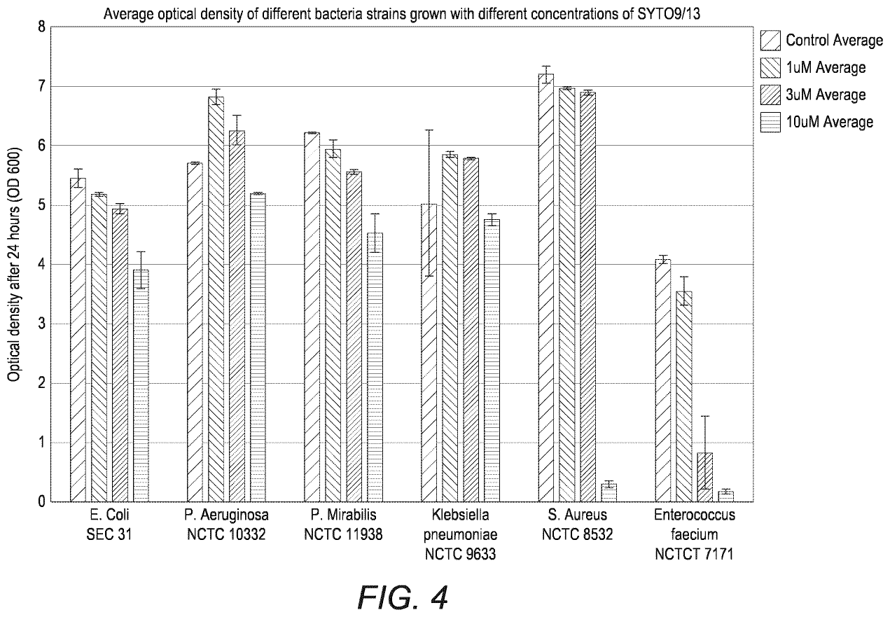

FIG. 4 is a graph showing is a graph showing the average optical density after 24 hours (OD 600) of P. Aeruginosa NCTC 10332, E. Coli SEC 31, Enterococcus faecium NCTCT 7171, P. Mirabilis NCTC 11938, S. Aureus NCTC 8532 and Klebsiella pneumoniae NCTC 9633, when incubated with DiSC3(5), SYBR Green I and SYTO 13/9 where different concentrations of SYTO 13/9 were assessed.

### EXAMPLE 1

Experiments were conducted to establish whether the combination of a number of dyes could be used to differentiate bacterial strains (P. Aeruginosa NCTC 10332, E. Coli SEC 31, Enterococcus faecium NCTCT 7171, P. Mirabilis NCTC 11938, S. Aureus NCTC 8532 and Klebsiella pneumoniae NCTC 9633) on the basis of fluorescent signal alone. The bacterial strains selected are the same or similar to those most widely reported in UTIs.

The following protocol was undertaken during these experiments.

Firstly, all different bacterial strains were grown overnight in LB at 37° C. and 200 rpm. The mid-exponential phase cultures of bacterial strains were then diluted to a concentration of 10⁵ cells·mL⁻¹ in LB. SYBR Green I, SYTO 13/9 and DiSC3(5) dyes were prepared by diluting them from their stocks.

The three dyes (SYBR Green I, SYTO 13/9 and DiSC3(5)) were then added in the final concentrations of 3 μM—DiSC3(5), 3 μM—SYTO 13/9 and 10× (diluted from the stock 10,000×)—SYBR Green I to the different bacterial strains and the strains incubated in the dak at 37° C. for 10 minutes in the dark.

The bacterial strains were then put on a haemocytometer and visualised under a microscope with fluorescent filters corresponding to individual dyes. Images were taken for all the bacterial strains at the same intensity of the incident laser light and 10-15 images were taken for each of the bacterial strains.

The images were then analysed on ImageJ software by first converting the coloured images to monochromatic images, then selecting an area with cells and analysing the plot profile. The intensity of the peaks and background from the image was then recorded.

The results of experiments are summarised in Table 1 below and illustrated in FIG. 1.

TABLE 1

| | DiSC3(5) | | SYBR Green I | | SYTO 13/9 | |
|---|---|---|---|---|---|---|
| | Average | St Dev | Average | St Dev | Average | St Dev |
| P. Aeruginosa | 0.00 | 0.00 | 123.67 | 27.18 | 113.55 | 20.89 |
| E. Coli | 62.66 | 3.60 | 104.53 | 27.68 | 205.43 | 3.99 |
| Enterococcus | 142.28 | 16.53 | 162.27 | 16.26 | 113.17 | 25.93 |
| faecium | | | | | | |
| P. Mirabilis | 88.50 | 7.96 | 109.85 | 14.34 | 102.50 | 22.77 |
| S. Aureus | 184.80 | 19.25 | 196.52 | 3.89 | 157.67 | 23.85 |
| Klebsiella | 16.70 | 0.67 | 21.87 | 5.10 | 173.10 | 12.32 |
| pneumoniae | | | | | | |

FIG. 1 clearly shows that the fluorescent signal intensity (after the background signal has been subtracted) differs for each dye for each of the strain tested.

Table 2 below shows the movement observed in each of the strains using the different dyes.

TABLE 2

| | | | Movement | Movement | Movement |
|---|---|---|---|---|---|
| | Gram | Shape | DiSC3(5) | SYBR Green I | SYTO 13/9 |
| P. Aeruginosa | −ve | Rod | Slow | Slow | Slow movement in |
| | | | movement in | movement in | straight lines |
| | | | straight lines | straight lines | |
| E. Coli | −ve | Rod | Wiggles | Wiggles | Wiggles |
| Enterococcus | +ve | Circular; | Stationary, | Stationary, | Stationary, buds |
| faecium | | but | buds | buds | |
| | | appears | | | |
| | | in chains | | | |
| P. Mirabilis | −ve | Rod | Fast | Fast movement | Fast movement in |
| | | | movement in | in straight lines | straight lines |
| | | | straight lines | | |
| S. Aureus | +ve | Circular; | Very stationary | Very stationary | Very stationary |
| | | in | | | |
| | | clusters | | | |
| Klebsiella | −ve | Rod | Stationary | Stationary | Stationary |
| pneumoniae | | | | | |

P. Aeruginosa NCTC 10332 had a fluorescence intensity profile (with the highest intensity listed first) of SYBR Green I>SYTO 13/9.

E. Coli SEC 31 had a fluorescence intensity profile (with the highest intensity listed first) of SYTO 13/9>SYBR Green I>DiSC3(5).

Enterococcus faecium NCTCT 7171 had a fluorescence intensity profile (with the highest intensity listed first) of SYBR Green I>DiSC3(5)>SYTO 13/9.

P. Mirabilis NCTC 11938, had a fluorescence intensity profile (with the highest intensity listed first) of SYBR Green I>SYTO 13/9>DiSC3(5).

S. Aureus NCTC 8532 had a fluorescence intensity profile (with the highest intensity listed first) of SYBR Green I>DiSC3(5)>SYTO 13/9 and whilst similar in profile to Enterococcus faecium NCTCT 7171, the overall intensity was greater.

Klebsiella pneumoniae had a fluorescence intensity profile (with the highest intensity listed first) of SYTO 13/9>SYBR Green I>DiSC3(5) and whilst similar in profile to E. Coli SEC 31, the overall intensity of SYBR Green I and DiSC3(5) was much less.

This data shows that a combination of SYBR Green I, SYTO 13/9 and DiSC3(5) dyes can enable clear differentiation of a number of bacterial strains. Advantageously, the combination of dyes also allowed for the differentiation of differently shaped bacteria and between Gram positive and Gram negative bacteria.

### EXAMPLE 2

Experiments were conducted to establish whether the combination of the DNA staining fluorescent dyes used in Example 1 could be used in conjunction with a membrane dye to help better differentiate bacterial strains (P. Aeruginosa NCTC 10332, E. Coli SEC 31, Enterococcus faecium NCTCT 7171, P. Mirabilis NCTC 11938, S. Aureus NCTC 8532 and Klebsiella pneumoniae NCTC 9633) on the basis of fluorescent signal alone. The bacterial strains selected were the same as those described in Example 1.

A similar protocol to Example 1 was followed, with the addition of 15 μm of FM 4-64.

Table 3 below and FIG. 2 shows the results of the experiments.

TABLE 3

| Strain | | DiSC3(5) 3 uM | | SYBR Green 10x | | SYTO 13/9 3 uM | | FM 4-64 15 uM | |
|---|---|---|---|---|---|---|---|---|---|
| # | Strain | Average | Std Dev | Average | Std Dev | Average | Std Dev | Average | Std Dev |
| 1 | P. Aeruginosa NCTC 10332 | 0.00 | 0.00 | 123.67 | 27.18 | 113.55 | 20.89 | 10.00 | 3.74 |
| 4 | E. Coli SEC 31 | 62.66 | 3.60 | 104.53 | 27.68 | 205.43 | 3.99 | 56.50 | 7.86 |
| 5 | Enterococcus faecium NCTCT 7171 | 142.28 | 16.53 | 162.27 | 16.26 | 113.17 | 25.93 | 25.00 | 2.50 |
| 10 | P. Mirabilis NCTC 11938 | 88.50 | 7.96 | 109.85 | 14.34 | 102.50 | 22.77 | 0.00 | 0.00 |
| 11 | S. Aureus NCTC 8532 | 184.80 | 19.25 | 196.52 | 3.89 | 157.67 | 23.85 | 89.00 | 6.85 |
| 12 | Klebsiella pneumoniae NCTC 9633 | 16.70 | 0.67 | 21.87 | 5.10 | 173.10 | 12.32 | 30.00 | 1.53 |

The results show that the inclusion of the FM 4-64 membrane dye did allow for better differentiation than relying upon DNA stain dyes alone.

### EXAMPLE 3

Comparative experiments were conducted using multiple DNA dyes in low concentration and one DNA at different (and higher) concentration in order to establish whether increasing DNA dye concentration would affect bacterial growth and hinder the differentiation of bacterial strains.

Experiments were conducted which were similar to Example 1.

In one experiment, 3 μM of DiSC3(5) and 3 μM of SYTO 13/9 was assessed with CYBR Green I at the following concentrations: 1× average, 3× average, 10× average, 30 average and 100× average.

In another experiment, 3 μM of DiSC3(5) and 10× of CYBR Green I was assessed with SYTO 13/9 at the following concentrations: 1 μM average, 3 μM average and 10 μM average.

Table 4 below and FIG. 3 shows the results of the experiments where the SYBR Green I dye was assessed at different concentrations relative other same concentrations of dyes of Example 2.

TABLE 4

| | Control | | 1x | | 3x | | 10x | | 30x | | 100x | |
|---|---|---|---|---|---|---|---|---|---|---|---|---|
| | | Std | | Std | | Std | | Std | | Std | | Std |
| | Average | dev | Average | dev | Average | dev | Average | dev | Average | dev | Average | dev |
| E. Coli SEC 31 | 4.572 | 0.62 | 3.407 | 0.39 | 3.306 | 0.56 | 3.139 | 0.29 | 2.086 | 0.32 | 2.514 | 0.97 |
| P. Aeruginosa NCTC 10332 | 5.785 | 0.45 | 5.785 | 0.82 | 5.785 | 0.39 | 2.968 | 0.44 | 3.113 | 0.59 | 2.39 | 0.08 |
| P. Mirabilis NCTC 11938 | 5.785 | 0.87 | 5.785 | 0.63 | 5.785 | 0.29 | 4.635 | 0.53 | 4.044 | 0.80 | 0.001 | 0.00 |
| Klebsiella pneumoniae NCTC 9633 | 3.689 | 0.14 | 4.719 | 0.14 | 4.907 | 0.21 | 3.439 | 0.62 | 2.053 | 0.30 | 1.138 | 0.07 |
| S. Aureus NCTC 8532 | 3.475 | 0.16 | 3.796 | 0.45 | 0 | 0.00 | 0 | 0.00 | 0 | 0.00 | 0 | 0.00 |
| Enterococcus faecium NCTCT 7171 | 3.906 | 0.13 | 1.158 | 0.00 | 1.315 | 0.04 | 0.136 | 0.02 | 0 | 0.00 | 0.022 | 0.00 |

Table 5 below and FIG. 4 shows the results of the experiments where the SYTO 9/13 dye was assessed at different concentrations relative other same concentrations of dyes of Example 2.

TABLE 5

| | Control | | 1 uM | | 3 uM | | 10 uM | |
|---|---|---|---|---|---|---|---|---|
| | Average | Std dev | Average | Std dev | Average | Std dev | Average | Std dev |
| E. Coli SEC 31 | 5.45 | 0.15 | 5.18 | 0.03 | 4.94 | 0.08 | 3.89 | 0.31 |
| P. Aeruginosa NCTC 10332 | 5.71 | 0.01 | 6.82 | 0.12 | 6.26 | 0.24 | 5.20 | 0.01 |
| P. Mirabilis NCTC 11938 | 6.21 | 0.01 | 5.94 | 0.15 | 5.56 | 0.03 | 4.53 | 0.33 |
| Klebsiella pneumoniae NCTC 9633 | 5.03 | 1.22 | 5.84 | 0.04 | 5.79 | 0.01 | 4.75 | 0.10 |
| S. Aureus NCTC 8532 | 7.19 | 0.14 | 6.97 | 0.02 | 6.89 | 0.05 | 0.30 | 0.06 |
| Enterococcus faecium NCTCT 7171 | 4.08 | 0.06 | 3.55 | 0.25 | 0.82 | 0.61 | 0.18 | 0.03 |

The results clearly showed using high concentrations of DNA dyes slowed bacterial growth and therefore using multiple DNA staining dyes at lower concentrations not only allowed for good optical differentiation of the bacterial strains but also did not slow the growth of the bacteria. The ability to allow for good optical differentiation and for bacterial growth is advantageous when assessing the ability of bacterial strains to grow in the presence or absence of an antimicrobial agent.

### EXAMPLE 4

Further comparative experiments were conducted using the dyes: SYBR Green I, DiSC3(5), FM4-64, and SYTO 13. The aim of these experiments was to determine the threshold value of fluorescence for each dye for a number of bacteria strains commonly associated with urinary tract infection. Once the threshold value for each dye in each strain was determined a set of unknown samples were tested in order to determine which microbial strain was present in each sample. The samples were chosen to be representative of patient samples.

The dye solutions were made up as detailed above and the bacterial strains were grown and then incubated with the dyes according to the following method.

### Bacterial Culture Preparation in Urine

An overnight culture was prepared by inoculating a bacterial colony in approximately 5 ml of Urine and incubating the culture in an orbital shaker at 37° C.

### Media/Materials Needed

The following media and materials were utilised: Cation-adjusted Mueller Hinton broth (MHB); Phosphate Buffered Saline (PBS); Membrane staining dye—FM4-64 (1.5 mM); Live cell staining dye—DiSC3(5) (1 mM); DNA staining dye—SYBR Green (1000×); DNA staining dye—SYTO 13 (0.5 mM); Ultrapure Agarose; Parafilm; Glass slides and coverslips; Scalpel; and EVOS M7000 fluorescent microscope.

### Agarose Pads Preparation

A pre-cut parafilm or a coverslip was used as a guide and 22 mm×22 mm squares cut from parafilm. A square was cut out of the centre of the parafilm (approximately 10 mm×10 mm). The centre of the cut-out was discarded. Two parafilm gaskets were placed onto a cleaned glass slide. The glass slide was then placed on a dry bath set to 56° C. so that the parafilm adhered to the glass slide. The glass slide was then kept on the dry bath. Using a microwave, ultrapure agarose was dissolved in PBS at a concentration of 1.2%. The agarose solution was kept on a dry bath set at 56° C. 60 μl of the agarose solution was pipetted in the centre of the parafilm gaskets. A coverslip was then placed over the gaskets without applying pressure on the coverslip. The slide was then placed on a cool surface and left to solidify for 1-2 minutes. The coverslips were then gently lifted off the agarose pads.

### Sample Preparation/Imaging

In a first Eppendorf, 15 μl of FM4-64 and 6 μl of SYTO 13 was added and in a second Eppendorf 10 μl of SYBR Green and 3 μl of DiSC3(5) was added. Depending on the bacterial growth in urine, an appropriate concentration of the bacterial culture was placed in the two Eppendorfs (final bacterial concentration 10⁶ CFU/ml). MHB was added so as to achieve a final volume of 1 ml. Final concentrations of the dyes in the sample was as follows: DiSC3(5): 3 μM; SYBR Green I: 10× (originally supplied as 10,000×); SYTO 13: 3 μM; and FM4-64: 15 μM

4 μl of the contents of each Eppendorf were deposited on one agarose pad and the cells distributed across the agar surface by tilting the slide. The slide was left to dry for approximately 1 minute. A coverslip was then placed on top of the agarose pad. The slide was placed inside the microscope and incubated at 37° C. for 10 minutes. Using different light filters for each dye, an image of each pad at 40× magnification on an EVOS M7000 fluorescent microscope was captured. Eight images for each agarose pad was recorded (i.e. 8 each for brightfield and 2 fluorescent channels). For each agarose pad, 24 images were recorded. This corresponded to 48 images in total for each bacterial strain (2 agarose pads per strain). Table A below was used to take the images using the correct fluorescent channel and intensity (for imagining on any other instrument, the LED intensity will need to be normalised against the EVOS M7000).

TABLE A

| | Excitation | Emission | |
|---|---|---|---|
| | wavelength (nm) / | wavelength (nm) / | LED intensity |
| | bandwidth | bandwidth | (as on EVOS 7000) |
| DiSC3(5) | 635/18 | 692/40 | 0.0065 |
| SYBR Green I | 482/25 | 524/24 | 0.20 |
| SYTO 13 | 482/25 | 524/24 | 0.20 |
| FM 4-64 | 542/20 | 692/40 | 0.051 |
| Brightfield | | | 0.079 |

Table 6 below shows the initial results of the dye thresholding experiment. The results are qualitative.

TABLE 6
Results of the initial dye thresholding experiment.

| | Fluorescence spectra data (thresholds) | | | | | | | |
|---|---|---|---|---|---|---|---|---|
| | SYBR Green | | DiSC3(5) | | FM4-64 | | SYTO 13 | |
| | Relative | Percentage | Relative | Percentage | Relative | Percentage | Relative | Percentage |
| | fluorescent | of cells | fluorescent | of cells | fluorescent | of cells | fluorescent | of cells |
| Strain name | intensity | stained | intensity | stained | intensity | stained | intensity | stained |
| Entercoccus | Medium-High | Almost all | Low | Half | Medium | Almost all | Medium | Almost all |
| Staphylococcus aureus | High | Almost all | High | Almost all | Medium-high | Almost all | High | Almost all |
| Staphylococcus epidermis | High | Almost all | Very high | Almost all | Medium | Almost all | High | Almost all |
| Staphylococcus saprophyticus | High | Almost all | High | Almost all | High | Almost all | Medium | Almost all |
| Streptococcus agalactiae | Very high | Almost all | Low | Almost all | High | Almost all | High | Almost all |
| Escherichia coli | Medium | Almost all | Medium | Almost all | Medium | Almost all | Medium | Almost all |
| Klebsiella | Low | Few | Low-Medium | Few | Low | Few | Low | Few |
| Proteus | Low | Half | Low-Medium | Half | Negligible | Few | Low | Half |
| Pseudomonas aeruginosa | Low | Almost all | Negligible | Few | Low | Almost all | Low | Half |

Note:
Bold cells are for major differentiators

Table 6 contains the relative fluorescent intensity threshold for each dye in each bacteria strain. The data shows the range of fluorescent intensity and the percentage of cells stained for each bacterial strain when stained with a specific fluorescent dye. The parameters in bold are those parameters needed to differentiate the bacterial strain from other strains. Any bacterial strain that doesn't show fluorescence parameters in any of the ranges in Table 6 is considered a non-UTI causative bacterial strain.

To qualitatively calculate the relative fluorescent intensities, cell size was compared between brightfield and the fluorescent channel for a given fluorescent dye. If the cells are very fluorescent, they will appear bigger in the fluorescent channel as compared to the bright field. For our qualitative measurements, if the cells looked bigger in the fluorescent channel, it was marked as having “high” relative fluorescent intensity. If the cells looked twice as big in the fluorescent channel, it was marked as having “very high” relative fluorescent intensity. If the cells looked almost the same size in the fluorescent channel and the bright field, it was marked as having “medium” relative fluorescent intensity. If the cells looked smaller in the fluorescent channel as compared to the brightfield but still the whole cell was visible, it was marked as having “low” relative fluorescent intensity. If only a few traces of a cell was visible in the fluorescent channel, it was marked as having “negligible” relative fluorescent intensity.

Enterococcus spp. shows medium to high (saturated; due to fluorescent saturation, the cells look bigger on the fluorescent channel as compared to the brightfield) fluorescence with SYBR Green I, low fluorescence with DiSC3(5) and medium fluorescence with FM 4-64 and SYTO 13.

Staphylococcus aureus shows high (saturated) fluorescence with SYBR Green I and high fluorescence (saturated) with DiSC3(5)

Staphylococcus epidermis shows very high (overly saturated) fluorescence with DiSC3(5) and high fluorescence (saturated) with SYTO13.

Staphylococcus saprophyticus shows high (saturated) fluorescence with DiSC3(5) and medium fluorescence (not saturated) with SYTO13.

Streptococcus agalactiae shows very high fluorescence with SYBR Green I and low fluorescence with DiSC3(5).

Escherichia coli shows relatively medium staining in every dye and for almost all cells.

Klebsiella shows very low staining with all fluorescent dyes, however every dye does stain a few cells.

Proteus shows no staining or negligible with FM 4-64 while other dyes stain the cells.

Pseudomonas aeruginosa shows no or negligible staining with DiSC3(5).

The thresholds above were then tested by staining bacterial strains taken from a commercial library. The results are shown below in Table 7.

TABLE 7
threshold data when commercially available strains were tested.

| | Fluorescence spectra data | | | | | | | |
|---|---|---|---|---|---|---|---|---|
| | SYBR Green | | DiSC3(5) | | FM4-64 | | SYTO 13 | |
| | Relative | Percentage | Relative | Percentage | Relative | Percentage | Relative | Percentage |
| | fluorescent | of cells | fluorescent | of cells | fluorescent | of cells | fluorescent | of cells |
| Strain name | intensity | stained | intensity | stained | intensity | stained | intensity | stained |
| Enterococcus faecalis NCTCT 12697 | Medium | Almost all | Low | Half | Medium | Almost all | Medium | Almost all |
| Enterococcus faecium NCTC 7171 | High | Almost all | Low | Almost all | Medium | Half | Medium | Almost all |
| Enterococcus faecium NCTC 13923 | High | Almost all | Low | Almost all | Medium | Half | Medium | Almost all |
| S. aureus NCTC 8532 | High | Almost all | High | Almost all | Medium | Almost all | High | Almost all |
| S. Aureus NCTC 9369 | High | Almost all | High | Almost all | High | Almost all | High | Almost all |
| S. saprophyticus NCTC 13634 | High | Almost all | High | Almost all | High | Almost all | Medium | Almost all |
| Streptococcus agalactiae NCTC 11239 | Very high | Almost all | Low | Almost all | High | Almost all | High | Almost all |
| E. coli SEC 93 | Medium | Almost all | Medium | Almost all | Medium | Almost all | Medium | Almost all |
| E. coli SEC 27 | Medium | Almost all | Medium | Almost all | Medium | Almost all | Medium | Almost all |
| Klebsiella pneumoniae NCTC 9633 | Low | Half | Low | Half | Low | Few | Low | Half |
| Klebsiella pneumoniae NCTC 14335 | Low | Almost all | Low | Almost all | Low | Half | Low | Half |
| Proteus mirabilis NCTC 11938 | Low | Half | Low | Almost all | Negligible | Few | Low | Almost all |
| Proteus mirabilis NCTC 10975 | Low | Half | Medium | Almost all | Negligible | Few | Low | Half |
| Pseudomonas aeruginosa NCTC 10332 | Low | Almost all | Negligible | Few | Negligible | Almost all | Low | Almost all |
| Pseudomonas aeruginosa NCTC 13715 | Low | Almost all | Negligible | Few | Low | Almost all | Low | Few |

From this data it was clear that by using the thresholds previously developed it was possible to differentiate the different bacterial strains from one another.

To further test the thresholds and ability to differentiate between different bacterial strains a set of samples intended to mimic clinical samples were tested using the four dyes and thresholds above. The results of this are shown below in Table 8.

TABLE 8
results obtained when samples representing clinical samples were tested using the dyes and thresholds described above.

| | Fluorescence spectra | | | | | | | |
|---|---|---|---|---|---|---|---|---|
| | SYBR Green | | DiSC3(5) | | FM4-64 | | SYTO 13 | |
| | Relative | Percentage | Relative | Percentage | Relative | Percentage | Relative | Percentage |
| | fluorescent | of cells | fluorescent | of cells | fluorescent | of cells | fluorescent | of cells |
| Strain name (internal code) | intensity | stained | intensity | stained | intensity | stained | intensity | stained |
| Enterococcus faecalis (001) | Medium | Almost all | Low | Half | Medium | Almost all | Medium | Almost all |
| Enterococcus faecalis (002) | Medium | Almost all | Low | Half | Medium | Almost all | Medium | Almost all |
| Enterococcus faecalis (003) | Medium | Almost all | Low | Half | Medium | Almost all | Medium | Almost all |
| Enterococcus faecium (011) | High | Almost all | Low | Almost all | Medium | Half | Medium | Almost all |
| Enterococcus faecium (012) | High | Almost all | Low | Almost all | Medium | Half | Medium | Almost all |
| Enterococcus faecium (013) | High | Almost all | Low | Half | Medium | Almost all | Medium | Almost all |
| Staphylococcus aureus (041) | High | Almost all | High | Almost all | High | Almost all | High | Almost all |
| Staphylococcus aureus (042) | High | Almost all | High | Almost all | Medium | Almost all | High | Almost all |
| Staphylococcus aureus (043) | High | Almost all | High | Almost all | Medium | Almost all | High | Almost all |
| Staphylococcus epidermis (044) | High | Almost all | Very high | Almost all | Medium | Almost all | High | Almost all |
| Staphylococcus epidermis (045) | High | Almost all | Very high | Almost all | Medium | Almost all | High | Almost all |
| Staphylococcus epidermis (046) | High | Almost all | Very high | Almost all | Medium | Almost all | High | Almost all |
| Staphylococcus saprophyticus (047) | High | Almost all | High | Almost all | High | Almost all | Medium | Almost all |
| Staphylococcus saprophyticus (048) | High | Almost all | High | Almost all | High | Almost all | Medium | Almost all |
| Staphylococcus saprophyticus (049) | High | Almost all | High | Almost all | High | Almost all | Medium | Almost all |
| Streptococcus agalactiae (081) | Very high | Almost all | Low | Almost all | High | Almost all | High | Almost all |
| Streptococcus agalactiae (082) | Very high | Almost all | Low | Almost all | High | Almost all | High | Almost all |
| Streptococcus agalactiae (083) | Very high | Almost all | Low | Almost all | High | Almost all | High | Almost all |
| Cory. Urealyticum (1001) | Negligible | Few | Negligible | Few | Negligible | Few | Negligible | Few |
| Cory. Urealyticum (1002) | Negligible | Few | Negligible | Few | Negligible | Few | Negligible | Few |
| Escherichia coli (051) | Medium | Almost all | Medium | Almost all | Medium | Almost all | Medium | Almost all |
| Escherichia coli (052) | Medium | Almost all | Medium | Almost all | Medium | Almost all | Medium | Almost all |
| Escherichia coli (053) | Medium | Almost all | Medium | Almost all | Medium | Almost all | Medium | Almost all |
| Klebsiella oxytoca (091) | Low | Almost all | Medium | Almost all | Low | Few | Low | Half |
| Klebsiella oxytoca (092) | Low | Almost all | Low | Almost all | Low | Almost all | Low | Almost all |
| Klebsiella oxytoca (093) | Low | Half | Low | Half | Low | Half | Low | Half |
| Klebsiella pneumoniae (094) | Low | Half | Low | Half | Low | Few | Low | Half |
| Klebsiella pneumoniae (095) | Low | Almost all | Low | Almost all | Low | Few | Low | Half |
| Proteus mirabilis (021) | Low | Half | Low | Almost all | Negligible | Few | Low | Half |
| Proteus mirabilis (022) | Low | Half | Medium | Almost all | Negligible | Few | Low | Almost all |
| Proteus mirabilis (023) | Low | Half | Medium | Almost all | Negligible | Few | Low | Half |
| Proteus vulgaris (024) | Low | Half | Low | Almost all | Negligible | Few | Low | Half |
| Proteus vulgaris (025) | Low | Half | Medium | Few | Negligible | Few | Low | Almost all |
| Proteus vulgaris (026) | Low | Almost all | Low | Almost all | Negligible | Few | Low | Almost all |
| Pseudomonas aeruginosa (031) | Low | Almost all | Negligible | Few | Low | Almost all | Low | Almost all |
| Pseudomonas aeruginosa (032) | Low | Almost all | Negligible | Few | Low | Almost all | Low | Half |
| Pseudomonas aeruginosa (033) | Low | Almost all | Negligible | Few | Low | Almost all | Low | Almost all |

The data in Table 8 shows that it is possible to differentiate a bacterial strain present in a clinical sample using the dyes and thresholds previously described. For example, it was possible to determine that the bacterial strain in samples 001, 002 and 003 was Enterococcus faecalis because the bacterial cells present in this sample showed high staining with SYBR Green I, FM4-64 and SYTO13 while lacking staining above the required threshold in DiSC3(5).

The forgoing embodiments are not intended to limit the scope of the protection afforded by the claims, but rather to describe examples of how the invention may be put into practice.

### REFERENCES

Åkerlund T, Nordström K, Bernander R: Analysis of cell size and DNA content in exponentially growing and stationary-phase batch cultures of Escherichia coli. J Bacteriol 1995; 177:6791-6797.
Baltekin Ö, Boucharin A, Tano E, Andersson D I, Elf J: Antibiotic susceptibility testing in less than 30 min using direct single-cell imaging. Proc Natl Acad Sci USA 2017; 114:9170-9175.
Choi J, Yoo J, Lee M, Kim E G, Lee J S, Lee S, Joo S, Song S H, Kim E C, Lee J C, Kim H C, Jung Y G, Kwon S: A rapid antimicrobial susceptibility test based on single-cell morphological analysis. Sci Transl Med 2014; 6:267ra174.
Kell D B, Kaprelyants A S, Weichart D H, Harwood C L, Barer M R: Viability and activity in readily culturable bacteria: a review and discussion of the practical issues. Antonie van Leeuwenhoek 1998; 73:169-187.
Kell D B, Oliver S G: How drugs get into cells: tested and testable predictions to help discriminate between transporter-mediated uptake and lipoidal bilayer diffusion. Front Pharmacol 2014; 5:231.
Kline K A, Lewis A L: Gram-Positive Uropathogens, Polymicrobial Urinary Tract Infection, and the Emerging Microbiota of the Urinary Tract. Microbiology spectrum 2016; 4.
Skarstad K, Boye E, Steen H B: Timing of Initiation of Chromosome Replication in Individual Escherichia coli cells. EMBO Journal 1986; 5:1711-1717.
Skarstad K, Steen H B, Boye E: Escherichia coli DNA Distributions Measured by Flow Cytometry and Compared with Theoretical Computer Simulations. J Bacteriol 1985; 163:661-668.
Steen H B: Flow cytometric studies of microorganisms. In Melamed M R, Lindmo T, Mendelsohn M L (eds.): Flow Cytometry and Sorting (2nd Edition). New York: Wiley-Liss Inc., 1990:605-622.
Steen H B, Boye E: Bacterial growth studied by flow cytometry. Cytometry 1980; 1:32-36.
Stokke C, Flåtten I, Skarstad K: An easy-to-use simulation program demonstrates variations in bacterial cell cycle parameters depending on medium and temperature. PLoS One 2012; 7:e30981.
Taheri-Araghi S, Brown S D, Sauls J T, McIntosh D B, Jun S: Single-Cell Physiology. Annu Rev Biophys 2015; 44:123-142.
Tandogdu Z, Wagenlehner F M: Global epidemiology of urinary tract infections. Curr Opin Infect Dis 2016; 29:73-79.
Wallden M, Fange D, Lundius E G, Baltekin Ö, Elf J: The synchronization of replication and division cycles in individual E. coli cells. Cell 2016; 166:729-739.

1. A combination of fluorescent dyes for identifying, or differentiating between, different bacterial strains, wherein the combination comprises two or more DNA staining fluorescent dyes and/or a cellular accumulation dye and/or a membrane staining dye and wherein the two or more DNA staining dyes and/or a cellular accumulation dye and/or membrane dye are selected from the following fluorescent dyes: SYBR Green I, SYTO 13 or 9, DiSC3(5), and/or FM4-64.

2. The combination according to claim 1, wherein the combination comprises FM4-64, SYBR Green I, SYTO 13 or 9, and DiSC3(5) and/or the SYTO 13 or 9 is present in a substantially similar amount to DiSC3(5).

3. (canceled)

4. The combination according to claim 1, wherein FM4-64 is present in an amount of about 15 μM and/or SYBR Green I is present in an amount of about 10× and/or SYTO 13 or 9 is present in an amount of about 3 μM and/or DiSC3(5) is present in an amount of about 3 μM.

5. The combination according to claim 1, wherein the combination is incorporated into a bacterial growth media and/or added to a biological sample.

6. (canceled)

7. The combination according to claim 1, wherein the combination is coated or provided in the chamber of a device for determining the susceptibility of a microorganism in an aqueous biological sample to an antimicrobial agent.

8. A method for identifying, or differentiating between, different bacterial strains in a biological sample, the method comprising:
a) contacting a dye combination of two of more DNA staining fluorescent dyes and/or a cellular accumulation dye and/or a membrane dye with an aqueous biological sample wherein the two or more DNA staining dyes and/or a cellular accumulation dye and/or membrane staining dye are selected from the following fluorescent dyes: SYBR Green I, SYTO 13 or 9, DiSC3(5), and/or FM4-64;
b) incubating the dye combination and the biological sample under conditions effective to enable staining of bacterial cells;
c) taking at least one image of the stained bacterial cells; and
d) qualitatively analysing the at least one image for fluorescent signal intensities for each of the two of more fluorescent dyes and comparing the fluorescent signal intensities with that of known fluorescent signal intensities for previously screened bacterial strains and identifying the bacterial strain in the biological sample when the fluorescent signal intensities are substantially similar to the fluorescent signal intensities of one of the previously screened bacterial strains.

9. The method according to claim 8, wherein the combination comprises FM4-64, SYBR Green I, SYTO 13 or 9, and DiSC3(5) and/or the SYTO 13 or 9 is present in a substantially similar amount to DiSC3(5).

10. (canceled)

11. The method according to claim 8, wherein FM4-64 is present in an amount of about 15 μM and/or SYBR Green I is present in an amount of about 10× and/or SYTO 13 or 9 is present in an amount of about 3 μM and/or DiSC3(5) is present in an amount of about 3 μM.

12. The method according to claim 8, wherein the dye combination is incorporated into a bacterial growth media and/or is in a desiccated form prior to being contacted with biological sample.

13. (canceled)

14. The method according to claim 8, wherein step b) further comprises incubating the biological sample in growth media under conditions effective to enable or encourage growth or proliferation of the bacterial cells and/or step b) further comprises heating the biological sample in the range of about 35° C. and about 40° C.

15. (canceled)

16. The method according to claim 8, wherein the biological sample is derived from an individual believed to be suffering from a bacterial infection and/or the biological sample is urine.

17-18. (canceled)

19. The method according to claim 8, wherein the stained bacterial cells are imaged using a microscope.

20. The method according to claim 14, wherein step c) comprises taking images of the stained bacterial cells continuously or periodically during incubation.

21. A kit for identifying, or differentiating between, different bacterial strains, in an aqueous biological sample, the kit comprising:
a) a combination of fluorescent dyes wherein the combination comprises two of more DNA staining dyes and/or a cellular accumulation dye and/or a membrane staining dye, wherein the two or more DNA staining dyes and/or a cellular accumulation dye and/or membrane staining dye are selected from the following fluorescent dyes: SYBR Green I, SYTO 13 or 9, DiSC3(5), and/or FM4-64;
b) at least one chamber for enabling the mixing of the dye combination with the biological sample so as to form a mixture; and
c) an imaging device for taking at least one image of the stained bacterial cells in the mixture and analysing the at least one image for fluorescent signal intensities for each of the two of more dyes and comparing the fluorescent signal intensities with that of known fluorescent signal intensities for previously screened bacterial strains and identifying the bacterial strain in the biological sample when the fluorescent signal intensities are substantially similar to the fluorescent signal intensities of one of the previously screened bacterial strainsbacterial strain.

22. The kit according to claim 21, wherein the dye combination comprises FM 4-64, SYBR Green I, SYTO 13 or 9, and DiSC3(5) and/or the SYTO 13 or 9 is present in a substantially similar amount to DiSC3(5).

23. (canceled)

24. The kit according to claim 22, wherein FM4-64 is present in an amount of about 15 μM and/or SYBR Green I is present in an amount of about 10× and/or SYTO 13 or 9 is present in an amount of about 3 μM and/or DiSC3(5) is present in an amount of about 3 μM.

25. The kit according to claim 21, wherein the dye combination is in a desiccated form and/or the chamber is coated in the dye combination and/or filled with the dye combination.

26-27. (canceled)

28. The kit according to claim 21, wherein the kit further comprises growth media and/or the kit further comprises a heating arrangement for heating the chamber.

29. (canceled)

30. The kit according to claim 21, wherein the kit further comprises two or more chambers and/or one or more antimicrobial agents.

31-32. (canceled)
